(19) United States
(12) Patent Application Publication
(10) Pub. No.: US 2018/0258007 A1
(43) Pub. Date: Sep. 13, 2018

(54) TUNGSTEN OXIDE PRIMER COMPOSITIONS

(71) Applicant: General Dynamics OTS - Canada, Inc., Repentigny (CA)

(72) Inventors: Benoit Jolicoeur, Terrebonne (CA); Marc Palardy, Ville Saint-Laurent (CA)

(21) Appl. No.: 15/539,635

(22) PCT Filed: Dec. 23, 2014
(86) PCT No.: PCT/CA2014/051265

§ 371 Date: Jun. 23, 2017

(51) Int. Cl. C06B 25/34; C06C 7/00; C06B 23/00; C01B 21/00; C07C 205/02; C01G 29/00; C08L 25/08

(52) U.S. Cl. CPC C06B 25/34; C06C 7/00; C06B 23/002; C06B 23/007; F42C 19/0803; C07C 205/02; C01G 29/00; C08L 25/08; C01B 21/00

(57) ABSTRACT

A primer composition is provided having a primary explosive and an oxidizer system containing at least one tungsten oxide or one tungstate compound. The oxidizer system can by non-hydroscopic and non-toxic. The primer can include reducing agents, sensitizers, binders and gas producing agents. The primer composition generally is applicable to any application or device that employs ignition of a propellant, a fuel, a relay charge, a delay charge, or a booster charge, including, but not limited to, air bag gas generator systems, signaling devices, ejection seats, small, medium or large arms ammunition primers, and the like.

### TECHNICAL FIELD

The technical field pertains to primer charges or mixes and more particularly relates to priming mixes intended for ammunition.

### BACKGROUND

Impact-sensitive prior formulations, which are frequently referred to as percussion priming compositions, primer composition, primer mixes, primer mixtures, priming mixes, priming mixtures or primer charges, generally consist of a primary explosive (highly sensitive explosive), a gas generating material, an oxidizer, a fuel, occasionally an abrasive material to increase sensitivity to initiation, and a binder. Most modern electric and percussion primers still contain lead styphnate or lead azide as primary explosive, antimony trisulfide as fuel, and barium nitrate as oxidizer in addition to several other ingredients. The presence of an oxidizer in priming mixes is necessary because of the poor oxygen content of lead based explosives. Toxicity of these conventional primer compositions causes expensive handling procedures for production and disposal. Moreover, contamination of firing ranges, both indoor and outdoor, causes military and civilian organizations around the world enormous remediation costs. In a joint effort to reduce these costs, governments and industries are currently developing non-toxic primers for ammunition dedicated to military and civilian applications.

Considerable efforts have been directed to removing the lead from primer compositions. However, less attention has been paid to barium nitrate which is also highly toxic and widely used as an oxidizer. See, for example, U.S. Pat. No. 5,684,268 and U.S. Pat. No. 5,831,208. A potential alternative is potassium nitrate, which has been found to perform as well as barium nitrate. However, such nitrate salts such as potassium nitrate, are very susceptible to atmospheric moisture due to their hygroscopicity. Consequently, failure rates for such primers are increased in high moisture environment conditions. Oxidizing agents such as chlorate and perchlorate salts are also toxic. Moreover, potassium chlorate is hygroscopic and corrosive to steel gun barrels. See U.S. Pat. No. 8,460,486.

Another potential alternative to avoid the use of heavy and toxic metals in primer compositions is the use of metastable interstitial composites (MIC). These compositions, also known as metastable nanoenergetic composites (MNC), nano-thermites or superthermites, typically include Al—MoO3, Al—WO3, Al—CuO, and Al—Bi2O3. In such composites, both the aluminum and oxidant powders have particulate sizes of less than 0.1 micron and more preferably 20 to 50 nanometers. The sensitivity and the high temperature output of the MIC are obtained by the high surface area of the particles. The surface area of particulates is proportional to the burning rate. The close proximity of the oxidizer and the reducing agent yields high friction sensitivity. However, MIC have been found difficult to handle due to their sensitivity to electrostatic discharge (ESD), friction, and their reactivity to air. This characteristic results in a large-scale manufacturing process which is less cost-effective and more hazardous than with conventional primer mixtures. See, for example, U.S. Pat. No. 5,717,159 and U.S. Pat. No. 7,670,446.

Therefore, it would be desirable to have a non-toxic, non-corrosive, non-hygroscopic, and safe to handle primer composition. Attempts have been made over the years to find a suitable oxidizing agent to meet these characteristics. As will be recognized by those skilled in the art, oxidizing agents for primer compositions should have low solubility, low hygroscopicity, high stability under various conditions, high heat of decomposition, good compatibility with other ingredients contained in the primer composition, as well as being a good oxygen donor. A primer mixture containing such an oxidizing agent would be suitable for a large variety of applications in the field of primer composition.

Accordingly, it is desirable to provide a priming composition having a wide range of ignition capabilities and addressing the foregoing and other related and unrelated problems in the art. In addition, other objects, desirable features and characteristics will become apparent from the subsequent summary and detailed description, and the appended claims, taken in conjunction with this background.

### SUMMARY

Briefly described, the present invention relates to a primer composition including at least one sensitive explosive, at least one fuel and at least one oxidant. Examples of applications and devices that can employ the primer composition of this invention include, but are not limited to, a seat belt tensioner, an air bag, a signal flare, a hand grenade, a mechanical launch grenade, a smoke grenade, a restraint system, an ejection seat, an explosive canopy, a drogue chute extractor, an aerial decoy, a powered hand tool, an industrial tool, a fastening device, a grenade launcher, a gas grenade, a stun grenade, a sub-munition, a projectile launcher, a pyrotechnic initiation delay device, an impulse motor, a delay detonator, a blasting cap, a rock crusher, a cable cutting device, a seismic explosive device, an explosive projectile, a shaped charge, a wellbore perforating apparatus, an anti-armor warhead, a muzzle-loading firearm, a burrowing animal exterminating device, a predator control device, an igniter for starting fires, an ignition system for chemical heaters, and the like.

U.S. Pat. No. 8,597,445 refers in column 4 to several U.S. patents disclosing and describing various applications and devices which can employ the primer composition of the present invention. Those applications and devices are given as examples, but are not limited to them.

In one embodiment, the primer compositions of the present invention include at least a primary explosive, a fuel and an oxidizer system comprising at least one form of tungsten oxide or tungstate compound. In greater detail, the oxidizing agent of the present invention may be selected from, but is not limited to, the group of tungsten oxides or tungstate compounds consisting of tungsten (III) oxide (W₂O₃), tungsten (IV) oxide (WO₂), tungsten (VI) oxide (WO₃), bismuth tungstate, aluminum tungstate, barium tungstate, calcium tungstate, cadmium tungstate, cupper tungstate, cobalt tungstate iron tungstate, lead tungstate, magnesium tungstate, nickel tungstate, sodium tungstate, potassium tungstate, lithium tungstate, manganese tungstate, strontium tungstate, zinc tungstate and zirconium tungstate, alone or in combination. Tungstate compounds generally relate to compounds that contain an oxanion of tungsten or a mix oxide containing tungsten, the simplest tungstate ion being WO42-.

In a further embodiment, the primer composition contains up to about 70% by weight of primary explosives, from about 15% to about 50% by weight of oxidizer, and from about 5% to about 30% by weight of a reducing agent. These primer compositions optionally may include approximately up to about 30% by weight of explosive sensitizer, up to about 25% by weight of a gas producing agent, up to about 20% by weight of friction agent, up to about 10% by weight of a decoppering agent and up to about 20% of a conductive component.

Generally, the primer composition of this invention can employ any primary explosive in combination with tungsten oxides or tungstate compounds. Examples of primary explosives that can be used include, but are not limited to, salts of trinitroresorcinol, dinitrobenzofuroxan (DNBF), potassium dinitrobenzofuroxane (KDNBF), diazodinitrophenol (DDNP, also known as DINOL), lead azide, silver azide, salts of fulminate, salts of hydrazoic acid, salts of 5-nitrotetrazole, tetrazene, salts of tetrazene, salts of amino guanidine, salts of cyanamide, nitrocyanamide salts, nitrophenol salts, nitrosophenol salts nitramine salts, salts of metazonic acid, oxalic salts, peroxides, acetylide salts, nitrogen sulphide, nitrogen selenide, thiocyanic salts, silver perchlorate, hexanitromannitol, and the like, including any combination thereof. The term salts is meant to include all salts that can function as primary explosives. The primary explosive of the present invention can be non-toxic and substantially free of lead, or can contain some lead compounds, such as lead containing explosives. The reducing agent, also called fuel or combustible, may be selected from, but is not limited to, aluminum, boron, silicon, titanium, tungsten, manganese, zirconium, bismuth sulfide, zinc sulfide, and combination thereof.

In a preferred embodiment, the primer composition contains from about 15% to about 50% by weight of DDNP as primary explosive, from about 25% to about 40% by weight of tungsten (IV) oxide, tungsten (VI) oxide and/or bismuth tungstate as oxidizer, from about 5% to about 20% by weight of zinc sulfide, zirconium hydride, titanium, aluminum and/or magnesium as reducing agent, from about 5% to about 20% of tetrazene as explosive sensitizer, from about 3% to about 15% of PETN as secondary explosive or gas producing agent, and from about 5% to about 15% of powdered glass, silicon carbide or calcium silicide as friction agent and from about 0% to about 10% of bismuth powder as decoppering agent. Weight percentages of all ingredients are based on the weights of dry ingredients.

The primer composition of the preferred embodiment utilizes oxidizing agents that are low in toxicity, free of heavy metals, stable to ageing, non-hygroscopic, and non-corrosive.

The combustion by-products of the preferred embodiment do not include heavy metal compounds of lead, barium, mercury, antimony, beryllium, cesium, cadmium, arsenic, chromium, selenium, strontium, tin, or thallium.

A further object of the present invention is to provide a primer composition which may be manufactured under conditions of high moisture without deleterious side chemical reactions. The primer mix is typically wet processed during production for safety reasons. The wet formed primer mix can then be rolled and loaded into primer cups. Variations in ingredients and their relative amounts result in chemical systems which possess sensitivity and output properties tailored to specific requirements.

The priming mixture according to the present invention was found to present a ballistic efficiency comparable to that of traditional lead styphnate mixtures, as well as fully within SAAMI or NATO standard performance limits, depending on the intended use. Moreover, the priming mixture of the invention functions excellently even at low temperatures, as required by NATO standards, and may therefore be used not only for practice or target range cartridges, like most known heavy-metal free primer compositions, but also for combat ammunitions and operational devices.

For better explaining the innovative principles of the present invention and the advantages it offers over the known art, embodiments of the priming mixtures applying the above principles will be described hereinafter.

### DETAILED DESCRIPTION

The present invention is mainly directed to priming compositions containing tungsten oxide compounds and their derivatives as oxidizers. In greater detail, the oxidizing agent may be selected from, but is not limited to, the group consisting of tungsten (III) oxide, tungsten (IV) oxide, tungsten (VI) oxide, bismuth tungstate, aluminum tungstate, barium tungstate, calcium tungstate, cadmium tungstate, cupper tungstate, cobalt tungstate iron tungstate, lead tungstate, magnesium tungstate, nickel tungstate, sodium tungstate, potassium tungstate, lithium tungstate, manganese tungstate, strontium tungstate, zinc tungstate and zirconium tungstate, alone or in combination. The priming mixtures of this invention, in addition to the oxidizer, include at least one primary explosive and may contain other priming components such as secondary explosives, gas producing, sensitizers, reducing agents, and friction agents.

This invention, even if it could be used in any apparatus or devices that requires a primer composition to ignite a propellant, a fuel, a relay charge, a delay charge, or a booster charge is specifically focused on primers for ammunition. As used herein, the terms small arms refers to a firearm that can be carried by a person and fired without any mechanical support and having a bore diameter generally of one inch or less. This invention of priming mixtures containing tungsten oxide and its derivatives as oxidizer can be utilized in any types of small caliber ammunition for small arms. This oxidizer may also be put in conventional primer compositions containing heavy metals or it could be included in low toxicity primer compositions. This term refers to priming mixtures containing no more than trace amounts of lead, barium, mercury, antimony, beryllium, cesium, cadmium, arsenic, chromium, selenium, strontium, tin and thallium. Furthermore, priming compositions containing tungsten oxide compounds and their derivatives as oxidizers may be used in electric primers comprising conductive or non-conductive primer compositions.

The priming mixtures of this invention can include from about 15% to about 50% by weight of an oxidizer system comprising tungsten oxides, tungstate, or their derivatives in combination with one or more other known oxidizer. It is expected that if the oxidizers of the present invention are mixed with any other known oxidizer, the chain reaction that allows the priming ignition will be engaged. The oxidizer system can include tungsten oxide or its derivatives alone or in combination with one or more other oxidizers. In more detail, examples of the second oxidizer include, but are not limited to, bismuth oxide, potassium nitrate, zinc peroxide, manganese dioxide, molybdenum trioxide, strontium nitrate, strontium peroxide, barium nitrate, tin oxide and iron oxide. In the preferred embodiment, the priming mixture systems can contain about 25% to about 40% by weight of an oxidizer system including tungsten (IV) oxide, tungsten (VI) oxide or bismuth tungstate. The oxidized system has a texture sufficient to enable the oxidizer system to flow when blended with the other ingredients such that the priming composition is substantially homogeneous.

In addition to a tungsten oxide or a tungstate compound oxidizer system, the priming mixtures include one or more primary explosives. Generally those oxidizers could be used with any primary explosives. Examples of primary explosives that can be used include, but are not limited to, salts of trinitroresorcinol, dinitrobenzofuroxan (DNBF), potassium dinitrobenzofuroxane (KDNBF), diazodinitrophenol (DDNP, also known as DINOL), lead azide, silver azide, salts of fulminate, salts of hydrazoic acid, salts of 5-nitrotetrazole, tetrazene, salts of tetrazene, salts of amino guanidine, salts of cyanamide, nitrocyanamide salts, nitrophenol salts, nitrosophenol salts nitramine salts, salts of metazonic acid, oxalic salts, peroxides, acetylide salts, nitrogen sulphide, nitrogen selenide, thiocyanic salts, silver perchlorate, hexanitromannitol, and the like, including any combination thereof. The term “salts” is meant to include all salts that can function as primary explosives. The priming mixtures of the present invention include one or more primary explosives in an amount up to 70% by weight. In the preferred embodiments, the priming mixtures contain from about 15% to about 50% by weight of a primary explosive such as DDNP.

The priming mixtures of the present invention also include one or more secondary explosives acting as sensitizer. This component will accelerate or modify the rate of the reaction in the primer and thereby increase the sensitivity. A large variety of sensitizers could be used in priming mixtures. In the present case, the sensitizer has been chosen in part because of its compatibility with the chosen primary explosive. In the preferred embodiment, tetrazene is selected as a secondary explosive to be combined with DDNP. Tetrazene, also known as tetracene, tetrazolyl guanyltetrazene hydrate or tetrazene-1-carboxamidine-4-(1-H-tetrazol-5-yl) monohydrate, is typically added to the mix in combination with DDNP to increase the sensitivity of the charge. In one embodiment, the second explosive, acting as sensitizer, is added to the mix in an amount from 0 to about 30% by weight of the priming mixture. In the preferred embodiment, the priming mixtures contain from about 5% to about 20% by weight of tetrazene.

In an exemplary embodiment, the priming mixture also includes other non-explosive sensitizers. Those sensitizers, also called friction agents, are usually inert substances that could increase the friction and impact sensitivities of the primer composition. Examples of sensitizers that can be used include, but are not limited to, powdered glass, silicon carbide, calcium silicide, iron sulfide, iron carbide, copper oxide, silicon nitride, and aluminum silicate. Typically the priming mixture contains from 0% to about 20% by weight of this material. In one particular embodiment, calcium silicide is added to the mixture in a range from about 5% to about 15% by weight.

To provide sources of expanding gas when the priming mixture is activated, a gas producing agent also is included to the mixture, in accordance with another exemplary embodiment. Single or double base propellant or secondary explosive such as pentaerythritol tetranitrate or trinitrotoluene could create this effect. Typically the priming mixtures contain from 0% to about 25% by weight of one or more gas producing agent. In the preferred embodiment, the priming mixture contains about 3 to about 15% by weight of PETN as gas producing agent.

The priming mixtures can include one or more reducing agents. This particular component may be added in the priming mixture in order to increase the heat of combustion. It may also be called the combustible or the fuel. It can be either a metallic or a non-metallic fuel or a combination of both. Examples of potential reducing agents comprises, but are not limited to, aluminum, zinc sulfide, magnesium, titanium, boron, zirconium hydride, calcium silicide, magnesium-aluminum alloy, silicon and zirconium. In general, the priming mixture of the present invention may contain from about 5% to about 30% by weight of such material. In some particular embodiments, the priming mixture incorporates from about 5% to about 20% by weight of zinc sulfide, zirconium hydride, titanium, aluminum, magnesium, or any combination thereof as reducing agent.

The priming mixtures can also include one or more decoppering agent. This agent is typically an additive dedicated to the primers intended to be used in ammunition. Most of the time, the decoppering agent is part of the propellant mix. However in some calibers, due to a special propellant or the absence of propellant, the decoppering agent may be added to the priming mixture. This substance is an additive known for the purpose of removing the copper build up from the internal surface of the gun bore. Examples of a decoppering agent include, but are not limited to, tin metal and compounds, bismuth metal and compounds or lead metal and compounds. In one embodiment, the priming mixture contains from 0% to about 10% by weight of bismuth.

The primer composition of the present invention may also contain up to about 2% of binder to minimize dusting, thus increasing safety during process. Typically, from about 0.5% to about 1.5% by weight is used in priming mixtures. The particular binder used will be selected, amongst other reasons, for its compatibility with the explosive formulation prepared. Binders to be used can be selected from a variety of gums such as gum arabics, guar gum or from a large variety of polymers. Such a binder may be in the form of any suitable water-soluble composition that does not react adversely with the other components of the invention and will perform its function of consolidating the primer mixture after drying. In the preferred embodiment, a styrene acrylic polymer is contained in the priming mixture in a range from about 0.5% to about 1.5% by weight.

A particular aspect of the invention, and the preferred one, involves a low toxicity primer composition using tungsten oxide, tungstate, or their derivatives as an oxidizer that can be used in training ammunition. In this type of priming composition every component is chosen for the absence of heavy metals. In this preferred embodiment, the primer does not contain more than trace amounts of lead, barium, mercury, antimony, beryllium, cesium, cadmium, arsenic, chromium, selenium, strontium, tin and thallium.

Typically, in this particular embodiment, the conventional lead-based primary explosive is replaced by DDNP, KDNBF or any other non-toxic primary explosive, alone or in combination. Those components are known to be substantially free of heavy metals and non-toxic. Generally, the components comprising a priming mixture will be selected or designed to have ballistic properties similar or better than priming formulations containing lead styphnate. In the preferred embodiment, the primer composition contains from about 15% to about 50% by weight of DDNP as a primary explosive coupled with from about 25% to about 40% by weight of tungsten (IV) oxide, tungsten (VI) oxide, bismuth tungstate, or a combination thereof as an oxidizer. As shown in further examples, this preferred composition gives results comparable to lead primers.

A particular application for primers with low toxicity of the present invention is its use in the class of ammunition which may be described as reduced-energy, or low energy ammunition. Such ammunition is used for training purposes. Examples of such training ammunition are described in U.S. Pat. Nos. 5,035,183 and 5,359,937.

The priming mixture of the present invention containing tungsten oxide, tungstate, or their derivatives as oxidizer can be utilized in those types of ammunition. The priming mixture, in some embodiments, can serve as an improved source of gases to be generated and directed to propel the projectiles. In another embodiment, it can serve to ignite a relatively small amount of propellant which will propel the projectile. In both cases, to be effective in the above-mentioned purposes, a primer composition for use in low energy ammunition should generate a substantial quantity of gas upon ignition. This output is achieved with the priming formulations of the present invention. In the preferred embodiment, a small amount of propellant will be used to insure sufficient chamber pressure is generated to cycle conventional small arms weapon mechanisms.

Because the projectile present in reduced energy ammunition, such as described in U.S. Pat. No. 5,359,937, is typically composed of a frangible shell containing a water-based marking compound there is an important focus put on the non-hygroscopicity and the chemical stability of the primer composition and most particularly to its oxidizer. The terms non-hygroscopic as used herein, generally refers to an article, compound or system that does not readily absorb and retain moisture, especially when exposed to humidity. The priming composition of the preferred embodiment, comprising tungsten (IV) oxide, tungsten (VI) oxide or bismuth tungstate, resists and is not affected by a particularly long storage time in humid conditions.

Primers according to the present invention incorporated into a low energy cartridge having a frangible bullet with a water-based marking compound have been stored in an airtight packaging during ageing trials. This configuration was able to perform adequately in ballistic tests after exposure to a high humidity environment for months. The weapon functioning results were comparable to cartridges utilizing lead-based priming compositions stored under the same conditions.

Another particular aspect of the invention is its use in electric primers. The present invention can be used in a variety of electrically initiated devices. Examples of such devices include, but are not limited to, bridgewire, exploding bridgewire, exploding foil initiator, semi-conductor bridge, laser ignition, induction, spark gap ignition, direct thermal contact, and the like. Additionally, the primer composition can include a sufficient amount of conductive material to enable its ignition by ohmic heating in electric primers. Conductive materials that can be used include, but are not limited to, graphite, acetylene black, carbon black, carbon nanotubes, and carbon fibers and the like, including any combination thereof. Examples of electrically initiated devices utilizing the present invention include, but are not limited to, those disclosed in U.S. Pat. No. 8,597,445; each of which is incorporated herein by reference in its entirety.

### Example 1

One embodiment of this invention is the use of low-toxicity primer in reduced-energy 9 mm training ammunition. Ballistic tests have been carried out to establish the following preferred weight percentages (Error! Reference source not found.) for a non-toxic primer composition according to the invention:

TABLE I

| | Preferred Min. | Preferred Max. |
|---|---|---|
| Component | Weight % | Weight % |
| Oxidizer | 25 | 40 |
| DDNP | 15 | 50 |
| Sensitizer | 5 | 20 |
| Reducing agent | 5 | 20 |
| Gas producing agent | 3 | 15 |
| Friction agent | 0 | 15 |
| Decoppering agent | 0 | 10 |
| Binder | 0.5 | 1.5 |

The above components are mixed as follows: the non-explosive components in the dry state are first mixed together; to this dry mix are added the explosive components (DDNP, tetrazene and pentaerythritol tetranitrate) maintained at such humidity that the final humidity of the mixture ranges between 10 and 15% by weight; finally, binder in water solution is added to the mixture. The resulting primer mixture is metered into primers comprising center-fire percussion caps for NATO 9 mm caliber cartridges, each comprising a cap and relative anvil and containing roughly 0.018 g of the prepared mixture. The primers are then fitted in a known manner to the above cartridges.

Using a damp mixture enables it to be metered more easily into the caps, and provides for maximum safety when preparing and processing the primers.

To illustrate that this tungsten oxide class of oxidizer were functioning in non-toxic priming compositions, different tungsten oxide and tungstate compounds were tried with the same mixture components. In the preferred embodiment, because of water-based marking composition within the frangible training projectile, only non-hygroscopic oxidizer have been selected (tungsten (IV) oxide, tungsten (VI) oxide, and bismuth tungstate). However, it is obvious that other hygroscopic tungstate compounds would give similar sensibility results before ageing. Three different mixes were prepared to illustrate the compatibility and capability of each oxidizer with other primer components. Mixture descriptions are found in Table II.

TABLE II
Percentage by weight of dry ingredients

| | EX1 | EX2 | EX3 |
|---|---|---|---|
| DDNP | 35 | 35 | 35 |
| Tetrazene | 15 | 15 | 15 |
| WO₂ | 35 | | |
| WO₃ | | 35 | |
| Bismuth tungstate | | | 35 |
| PETN | 5 | 5 | 5 |
| Al | 5 | 5 | 5 |
| Bi Binder | 5 | 5 | 5 |

Once mixed, these components were assembled in small arms primers. After drying, these primers were tested according to Sporting Arms and Ammunition Manufacturers' Institute (SAAMI) specification for small arms ammunition sensitivity. For 9 mm caliber primers, the accepted performance standard requires that the average height (HO), when a 1.94 ounce weight is dropped, minus 2 standard deviations (SD) must be greater to 1 inch and this average height (HO) plus 4 standard deviations (SD) must be less than 11 inches. The average height (HO) is the value where statistically 50% of primers fires.

The Table III shows sensitivity results for these priming mixtures in 9 mm caliber primers.

TABLE III

| | 25 samples tested at each level | | | SAAMI |
|---|---|---|---|---|
| | EX1 | EX2 | EX3 | requirement |
| H + 4SD (in) | 7.51 | 8.28 | 8.88 | ≤11.00 |
| H − 2SD (in) | 4.25 | 4.59 | 3.99 | ≥1.00 |
| H₀ (in) | 5 | 5 | 6 | — |

Table III demonstrates that although there are some differences in sensitivity between each oxidizer, these three samples show results within the SAAMI guidelines. Those three tungsten oxide oxidizers in a typical formulation therefore meet the SAAMI performance standard.

### Example 2

In order to demonstrate the versatility of the tungsten oxide compounds, this example reports the results obtained for tungsten oxide priming mixtures with a large variety of reducing agents and friction agents. Table IV presents many formulations comprising bismuth tungstate, WO2 and WO3 as oxidizer with some reducing agent or friction agent. The preferred embodiment was also used for this example.

TABLE IV
Percent by weight dry ingredients

| | EX4 | EX5 | EX6 | EX7 | EX8 | EX9 | EX10 | EX11 | EX12 |
|---|---|---|---|---|---|---|---|---|---|
| DDNP | 35 | 35 | 35 | 35 | 35 | 35 | 35 | 35 | 35 |
| Tetra- | 15 | 15 | 15 | 15 | 15 | 15 | 15 | 15 | 15 |
| zene | | | | | | | | | |
| Bismuth | 30 | 30 | 35 | 33 | 33 | 30 | | | |
| tung- | | | | | | | | | |
| state | | | | | | | | | |
| WO₂ | | | | | | | 30 | | |
| WO₃ | | | | | | | | 30 | 30 |
| PETN | 5 | 5 | 5 | 5 | 5 | 5 | 5 | 5 | 5 |
| Al | 10 | | | | 5 | 5 | | | |
| Ti | | | | 7 | | | | | |
| ZnS | | 10 | | | | | 10 | 10 | |
| ZrH₂ | | | 5 | | | | | | 10 |
| CaSi₂ | | | | | 2 | | | | |
| SiC | | | | | | 5 | | | |
| Bismuth | 5 | 5 | 5 | 5 | 5 | 5 | 5 | 5 | 5 |
| Binder | | | | | | | | | |

Once the primer formulations were assembled, they were dried and inserted in 9 mm caliber cartridge cases and further tested for their impact sensitivity according to SAAMI specifications. Results of the sensitivity testing are presented in Table V.

TABLE V
25 samples tested at each level

| | EX4 | EX5 | EX6 | EX7 | EX8 | EX9 | EX10 | EX11 | EX12 |
|---|---|---|---|---|---|---|---|---|---|
| H + | 9.07 | 7.88 | 7.86 | 8.71 | 7.93 | 9.40 | 7.93 | 8.96 | 7.26 |
| 4SD | | | | | | | | | |
| (in) | | | | | | | | | |
| H − | 3.89 | 3.77 | 3.96 | 3.47 | 4.34 | 4.27 | 4.34 | 3.05 | 4.32 |
| 2SD | | | | | | | | | |
| (in) | | | | | | | | | |
| H₀ | 6 | 5 | 5 | 5 | 5 | 6 | 5 | 5 | 5 |
| (in) | | | | | | | | | |

Each primer formulation met the SAAMI specification for impact sensitivity. Consequently, it is evident that tungsten oxide compounds perform well with a variety of friction agents or reducing agents. However, sensitivity is not the only criteria that a primer must meet. The ballistic norms of formulation were evaluated by loading the primers into 9 mm caliber “reduced-energy” cartridges. Those special munitions are not tested by the standard Electronic Pressure Velocity and Action Time (EPVAT) test that measure internal ballistics peak pressure, muzzle velocity, and action time. Instead they are evaluated on the projectile velocity and by weapon functioning defects. The average projectile velocity must be at least 140 m/s at 3 meters from the weapon muzzle and the defect rate must be less than 3.5% in many 9 mm caliber regular weapon types at three specific temperatures.

Table VI shows velocity and functioning test results of four of those formulations in comparison with a conventional lead primer. This comparison primer is a commercial CCI 500 small pistol primer with a lead styphnate primary explosive and a barium nitrate oxidizer. It has been selected because of its known sensitivity, good functionality and humidity resistance.

TABLE VI

| | CCI primer | EX1 | EX5 | EX10 | EX11 |
|---|---|---|---|---|---|
| Mean velocity (m/s) | 158 | 152 | 151 | 151 | 147 |
| Minimal velocity (m/s) | 147 | 146 | 144 | 142 | 141 |
| Maximal velocity (m/s) | 166 | 158 | 156 | 159 | 155 |
| % of defect | 0.8 | 0.8 | 1.7 | 1.7 | 0.8 |

Because it is an important matter in “reduced-energy” ammunition, resistance to ageing test were performed on those assembled primers. In order to prove primer composition stability over time, an accelerating ageing process was performed on cartridges by conditioning them for 3 months at 40° C. in hermetic barrier bags under a high humidity atmosphere. After this incubation period, the same ballistic measurements were performed. Table VII presents functioning results after accelerated ageing.

TABLE VII

| | CCI primer | EX1 | EX5 | EX10 | EX11 |
|---|---|---|---|---|---|
| Mean velocity | 146 | 140 | 140 | 139 | 138 |
| Minimal velocity | 130 | 136 | 134 | 129 | 132 |
| Maximal velocity | 157 | 147 | 147 | 151 | 144 |
| % of defect | 2.5 | 0 | 0.8 | 0.8 | 1.7 |

As expected, after accelerated ageing the projectile velocity has declined but the defect percentage is still within to the requirement confirming these formulations resistance to humidity. Those results are for informational purpose only because there is no requirement after ageing. The defect percentage is generated by 11 different types of defects in reduced energy marking ammunition. A loose primer in the pocket, broken sabot (a sabot is a plastic piece used in reduced-energy ammunition, as described in U.S. Pat. No. 5,359,937), or perforated primer cup represent some examples of the compiled defects. Those are highly modulated by the primer pressure developed. Considering that this pressure is higher before accelerated ageing, it is normal, if the primer retains its good functionality over time and does not deteriorate, to measure a lower rate of defect after ageing.

From the above data, the versatility and robustness of the tungsten oxides and tungstate compounds in a variety of priming formulations that can be used in reduced energy small arms training and service ammunition become apparent.

### Example 3

A series of primer composition were prepared as in Example 1 according to the preferred embodiment. Examples shown in Table VIII all contained 0.8% in excess by weight of styrene acrylate polymer as binder. These primer compositions were loaded in center-fire percussion caps for NATO .50 caliber (also known as 12.7 mm caliber) cartridges. After assembling and drying, they were comparison tested with traditional primers containing a proven lead styphnate priming mixture, known as FA-958, manufactured by the applicant.

TABLE VIII
Percentage by weight of dry ingredients

| | EX13 | EX14 | EX15 | EX16 | EX17 | FA-958 |
|---|---|---|---|---|---|---|
| Lead styphnate | | | | | | 28 |
| DDNP | 39 | 39 | 39 | 35 | 37 | |
| Tetrazene | 7 | 7 | 7 | 11 | 10 | 4 |
| Barium nitrate | | | | | | 39 |
| Bismuth tungstate | 32 | | | 32 | 35 | |
| WO₂ | | 32 | | | | |
| WO₃ | | | 32 | | | |
| PETN | 8 | 8 | 8 | 6 | 7 | 7 |
| Al | 14 | 14 | 14 | 14 | 11 | 10 |
| Antimony sulfide | | | | | | 12 |
| Bismuth | | | | 2 | | |

Testing comprised sensitivity test and EPVAT, both performed according to the NATO documents Manual Of Proof and Inspection (MOPI) Multi-Caliber and AC225 standard manual, and the results of which are shown in Table IX and Table X. For the purpose of this evaluation, primers were assembled in a .50 caliber Target Practice (TP) cartridge.

TABLE IX
25 samples tested at each levl

| | | | | | | FA-958 | NATO |
|---|---|---|---|---|---|---|---|
| | EX13 | EX14 | EX15 | EX16 | EX17 | primers | requirements |
| H + 5SD (in) | 10.59 | 12.65 | 13.32 | 12.84 | 13.37 | 13.20 | ≤14.96 |
| H − 2SD (in) | 4.36 | 5.38 | 5.68 | 5.31 | 5.09 | 7.24 | ≥2.48 |
| H₀ (in) | 6.14 | 7.46 | 7.86 | 7.46 | 7.46 | 8.94 | No |
| | | | | | | | requirement |

TABLE X
10 samples per example per temperature

| | | | | | NATO |
|---|---|---|---|---|---|
| | | | | FA-958 | Require- |
| | EX13 | EX15 | EX16 | primers | ments |
| Mean Case Mouth | 356 | 362 | 344 | 359 | ≤450 |
| Pressure + 3SD | | | | | |
| at +21° C. (MPa) | | | | | |
| Individual Case | ≤389 | ≤394 | ≤411 | ≤357 | ≤450 |
| Mouth Pressure | | | | | |
| at +52° C. | | | | | |
| and −54° C. (MPa) | | | | | |
| Mean Action | 3.4 | 3.7 | 3.7 | 3.3 | ≤4.5 |
| Time + 5SD | | | | | |
| at −54° C. (ms) | | | | | |

From the above examples, the performances of the tungsten oxides and tungstate compounds in a variety of priming formulations show that it can be used in conventional training and operational small arms ammunition.

Obviously, the above description of an embodiment applying the innovative principles of the present invention is taken by way of example only and therefore must not be considered as a limitation of the patent rights claimed herein. It will be obvious to those skilled in the art that many additions, deletions, and modifications can be made without departing from the spirit and the scope of this invention. No undue limits should be imposed, except for those set forth in the following claims.

Further, while at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure, it being understood that various changes may be made in the function and arrangement of elements described in an exemplary embodiment without departing from the scope as set forth in the appended claims and their legal equivalents.

What is claimed is:

1. A primer composition comprising:
a primary explosive;
an oxidizing agent; and
a reducing agent;
wherein the oxidizing agent consists essentially of tungsten oxides or tungstate compound.

2. A primer composition comprising:
a. no more than about 70% by weight of a percussion-sensitive organic primary explosive compound;
b. from about 15% to about 50% by weight of a oxidizer;
c. from about 5% to about 30% by weight of a reducing agent;
d. from 0% to about 30% by weight of a sensitizer;
e. from 0% to about 25% by weight of a gas producing agent;
f. from 0% to about 20% by weight of a friction agent;
g. from 0% to about 10% by weight of a decoppering agent; and
h. from 0% to about 20% by weight of a conductive component.

3. The primer composition of claim 2, wherein the percussion-sensitive organic primary explosive compound comprises a compound chosen from salts of trinitroresorcinol, dinitrobenzofuroxan (DNBF), potassium dinitrobenzofuroxane (KDNBF), diazodinitrophenol (DDNP), lead azide, silver azide, salts of fulminate, salts of hydrazoic acid, salts of 5-nitrotetrazole, tetrazene, salts of tetrazene, salts of amino guanidine, salts of cyanamide, nitrocyanamide salts, nitrophenol salts, nitrosophenol salts nitramine salts, salts of metazonic acid, oxalic salts, peroxides, acetylide salts, nitrogen sulphide, nitrogen selenide, thiocyanic salts, silver perchlorate, hexanitromannitol, and the like, including any combination thereof.

4. The primer composition of claim 2, wherein the oxidizer is a compound of tungsten oxide selected from the group consisting of tungsten (III) oxide, tungsten (IV) oxide, tungsten (VI) oxide, bismuth tungstate, aluminum tungstate, barium tungstate, calcium tungstate, cadmium tungstate, copper tungstate, cobalt tungstate iron tungstate, lead tungstate, magnesium tungstate, nickel tungstate, sodium tungstate, potassium tungstate, lithium tungstate, manganese tungstate, strontium tungstate, zinc tungstate, zirconium tungstate, and any combination thereof.

5. The primer composition of claim 2, wherein the reducing agent is chosen from aluminum, zinc sulfide, magnesium, titanium, boron, zirconium hydride, calcium silicide, magnesium-aluminum alloy, silicon, zirconium, and any combination thereof.

6. The primer composition of claim 2, wherein the sensitizer comprises tetrazene.

7. The primer composition of claim 2, wherein the gas producing agent comprises pentaerythritol tetranitrate, trinitrotoluene, or a combination thereof.

8. The primer composition of claim 2, wherein the friction agent is chosen from powdered glass, silicon carbide, calcium silicide, iron sulfide, iron carbide, copper oxide, silicon nitride, aluminum silicate, and any combination thereof.

9. The primer composition of claim 2, wherein the decoppering agent is chosen from tin metal and compounds, bismuth metal and compounds, and lead metal and compounds.

10. The primer composition of claim 2, wherein the conductive component is chosen from graphite, acetylene black, carbon black, carbon nanotubes, carbon fibers, and any combination thereof.

11. The primer composition of claim 2, further comprising no more than about 5% by weight of a binder.

12. A priming composition comprising:
a. from about 15% to about 50% by weight of a percussion-sensitive organic primary explosive compound;
b. from about 25% to about 40% by weight of a non-hygroscopic oxidizer;
c. from about 5% to about 20% by weight of a reducing agent/combustible/fuel;
d. from about 5% to about 20% by weight of a sensitizer;
e. from about 3% to about 15% by weight of a gas producing agent;
f. from about 5% to about 15% by weight of a friction agent; and
g. from 0% to about 10% by weight of a decoppering agent.

13. The priming composition of claim 12, wherein the percussion-sensitive organic primary explosive compound comprises a compound chosen from dinitrobenzofuroxan (DNBF), potassium dinitrobenzofuroxane (KDNBF), diazodinitrophenol (DDNP), and any combination thereof.

14. The priming composition of claim 12, wherein the non-hygroscopic oxidizer comprises a non-hygroscopic oxidizer compound of tungsten oxide selected from the group consisting of tungsten (III) oxide, tungsten (IV) oxide, tungsten (VI) oxide, bismuth tungstate, and any combination thereof.

15. The priming composition of claim 12, wherein the reducing agent/combustible/fuel is chosen from zinc sulfide, zirconium hydride, aluminum, magnesium, titanium, and any combination thereof.

16. The priming composition of claim 12, wherein the sensitizer comprises tetrazene.

17. The priming composition of claim 12, wherein the gas producing agent comprises pentaerythritol tetranitrate.

18. The priming composition of claim 12, wherein the friction agent is chosen from powdered glass, silicon carbide, calcium silicide, and any combination thereof.

19. The priming composition of claim 12, wherein the decoppering agent comprises a bismuth powder.

20. The priming composition of claim 12, further containing no more than about 5% by weight of a binder.

21. The priming composition of claim 20, wherein the binder comprises a styrene acrylic polymer compound.

22. The priming composition of claim 12, wherein the priming composition, when fired, produces residues that are free of toxic substances.

23. The priming composition of claim 22, wherein the priming composition, when fired, produces residues that are free of heavy metals, such as mercury, lead, barium, antimony, beryllium, cadmium, arsenic, chromium, selenium, tin and thallium.
